(19) United States
(12) Patent Application Publication
(10) Pub. No.: US 2020/0326318 A1
(43) Pub. Date: Oct. 15, 2020

(54) MARKERS FOR CONSUMER PRODUCTS OR INDUSTRIAL PRODUCTS

(71) Applicant: IN-CODE TECHNOLOGIES LTD, St Albans (GB)

(72) Inventor: Ruprecht KELLER, Schleiden (DE)

(21) Appl. No.: 16/756,636

(22) PCT Filed: Oct. 22, 2018
(86) PCT No.: PCT/EP2018/078936

§ 371 Date: Apr. 16, 2020

(30) Foreign Application Priority Data
Oct. 20, 2017 (EP) 17197462.9

(51) Int. Cl. G01N 31/22; G01N 33/58; G06Q 10/08

(52) U.S. Cl. CPC G01N 31/22; G06Q 10/083; G01N 33/58

(57) ABSTRACT

Provided is the use of a unique marker profile for labeling consumer products or industrial products, wherein the unique marker profile comprises one, two or more marker substances comprising one, two or more polyethylene glycols.

### TECHNICAL FIELD

The present invention is generally related to the use of a unique marker profile for labeling consumer products or industrial products for any purpose including, for example, to indicate the origin, and/or intended recipient, and/or a predetermined characteristic (e.g. geographic location) of an intended recipient of the products and/or to determine that the product has not been altered or tampered.

### BACKGROUND OF THE INVENTION

The authentication and tracking of consumer products or industrial products is becoming an increasing concern by governmental agencies, manufacturers, distributors, sellers, and consumers, for a variety of reasons. For example, there has been a recent proliferation of counterfeit consumer products or industrial products which are made to appear similar or even identical to legitimate products and often sold on the so-called “black market” or integrated into the normal stream of commerce, being passed off as legitimate. Counterfeiting also means tampering or dilution of the respective products, for example dilution of liquid products such as beverages or perfume.

Manufacturers, distributors, and/or sellers have an interest in tracking the source of legitimate consumer products or industrial products. It has been estimated that the growth rate of counterfeit branded goods exceeds economic growth rates worldwide. The ingredients of a counterfeit product may be the same as or may be different from those of the genuine product. Even if the ingredients are the same, they may be of inferior quality or may have been adulterated. Product adulteration is when a product is tampered with such as by dilution. An example of such a problem is in the liquor industry where bottles may be refilled or diluted with an inferior product in order to extend the product and to avoid paying taxes. In addition to the economic loss to the producers, counterfeit consumer products, such as beverages or food products, may contain harmful additives or contaminants, such as methanol in alcoholic beverages, to cause in extreme cases poisoning in consumers or even death of consumers. Thus, there is a need to track legitimate consumer products or industrial products to determine and detect the presence of fake goods. Labeling should be made in such a way that no complex analysis is necessary in order to analyze a sample of the consumer product for detection purposes.

In all of these circumstances, it is desirable to have a means of authenticating or determining the source of the consumer product or industrial product in question. In this context there is no question that any analytical investigation of a sample is only meaningful if the results obtained in the investigation can be used to determine if a product was made by a particular manufacturer (in the case of authenticating potential black market goods), determine what geographic market was intended for the product (in the case of authenticating potential grey market goods), determine whether the product has been altered or tampered or determine a particular factory or specific batch of origin (in the case of determining the origin of a consumer product or industrial product), in order to then initiate the correct response. An object of the present invention is to provide marker substances that can be used in low concentration and which can be directly detectable by suitable handheld devices (e.g. Raman spectrometers). A further object of the invention is the use of marker substances that can be used in the blockchain technology.

### SUMMARY OF THE INVENTION

The present invention provides the use of a unique marker profile for labeling consumer products or industrial products to indicate or trace any aspect of the consumer product or industrial product including, for example, its site of manufacture, intended recipient (e.g., geographical or other market for intended sale), and as a marker of authenticity.

In one aspect, the invention provides the use of a unique marker profile for labeling consumer products or industrial products, wherein the unique marker profile comprises one, two or more marker substances comprising one, two or more polyethylene glycols. The one, two or more marker substance(s) may be incorporated into the consumer product at any point during the manufacturing process according to known techniques appropriate for the particular consumer product and marker substance.

In another aspect, the invention provides a labeled consumer product or industrial product comprising:
a unique marker profile encoding one, two or more of the origin of the consumer product or industrial product, the intended recipient of the consumer product or industrial product, and a preselected characteristic of an intended recipient of the consumer product or industrial product;
wherein said unique marker profile comprises one, two or more polyethylene glycols.

In some embodiments of any of the aspects of the invention, the consumer product or industrial product contains at least two, three, four, five, or more marker substances.

In some embodiments the one, two or more marker substances further comprise one, two or more marker substances selected from carbohydrates, disaccharides, trisaccharides, tetrasaccharides, oligosaccharides, polysaccharides, isoprenoids, lipids, steroids, acrylic polymers, poloxamers, polyoxyls, polysorbates, acesulfame, an acetylated monoglyceride, butylparaben, povidone, copovidone, crospovidone, gelucire, hypromelloses, polycarbophil, polydextrose, tartaric acid or a salt thereof, and derivatives thereof.

In some embodiments, marker substances are substances identifiable or quantifiable by enzymatic, immunological, spectrometric, or electrophoretic methods and/or by an instrumental analytical chemistry technique including, for example, mass spectrometry (e.g., Gas Chromatography/Mass Spectrometry (GC/MS), Gas Chromatography/Tandem Mass Spectrometry (GC/MS/MS), High Performance Liquid Chromatography/Mass Spectrometry (HPLC/MS), High Performance Liquid Chromatography/Tandem Mass Spectrometry (HPLC/MS/MS), High Performance Liquid Chromatography (HPLC), Gas Chromatography (GC)), or Raman Spectroscopy.

In some embodiments, the unique marker profile comprises two or more marker substances, and at least two of said marker substances are the same type of marker substances. Alternatively, at least two of said marker substances are different types of marker substances.

In other preferred embodiments, the marker substances include a polydisperse polyethylene glycol and/or a monodisperse polyethylene glycol. Particularly useful marker profiles comprise two or more monodisperse polyethylene glycols each with different molecular weights.

In some embodiments, the unique marker profile indicates the origin of said consumer product or industrial product (e.g., the country, region, or manufacturing site (production facility)), the intended recipient of said consumer product or industrial product such as a specific customer or organization (such as UNO) to whom the product is to be shipped and/or a preselected characteristic of an intended recipient of said consumer product or industrial product (e.g., geographic region in which an intended recipient is located). Some of the embodiments described above recite a preselected characteristic of an intended recipient of a consumer product or industrial product. This characteristic may be any characteristic useful to group or categorize intended recipients. For example, the unique marker profile may include marker substances which indicate an intended recipient is found within a particular geographic region. In another example, the unique marker profile may include marker substances which indicate an intended recipient is subject to review by a particular regulatory or other governmental agency.

In some embodiments, the identification of the marker profile and its indication may be based on the identity of the marker substances alone (i.e., the presence or absence of the marker substances), the relative amounts of the marker substances (i.e., the ratios of the marker substances relative to each other in the consumer product or industrial product), the absolute amounts of the marker substances in the consumer product or industrial product, or some combination thereof.

As used herein, the term “origin of a consumer product or industrial product” is an absolute term denoting the location or facility where a consumer product or industrial product was manufactured or produced.

As used herein, the term “source of a consumer product or industrial product” is a relative term denoting the supplier of a consumer product or industrial product. In some instances, the source of a consumer product or industrial product may be the same as the place of origin, i.e., the location or facility of origin. In other instances, consumer products or industrial products may be shipped from their location or facility of origin to a recipient, who may then themselves become a source of the consumer product if they provide the consumer products or industrial products to another party.

As used herein, the term “counterfeit” or “counterfeiting” is to be understood as referring to products which are an imitation or copy of an original product, as well as referring to original products which have been tampered or altered, for example by dilution or replacement of some parts of the product.

As used herein, the term “derivative” is to be understood as all subsequent products which arise as a result of an induced or naturally occurring chemical transformation of a substance. Derivatives of marker substances can arise in the organism of the subject (e.g., by metabolism of the marker substance), or in a sample by induced or naturally occurring chemical transformation. Derivatives may also arise when used in the consumer product as such.

As used herein, the term “quantity” is to be understood as the amount of a particular substance and may be used to describe either an absolute measure of a substance in a sample, or to describe the relative amount of a substance in a sample with respect to some other substance(s) in the sample. Likewise, the term “quantify” is to be understood as to determine an absolute measure of a substance in a sample, or determine the relative amount of a substance in a sample with respect to some other substance(s) in the sample.

### DETAILED DESCRIPTION

The following description and examples describe in detail exemplary embodiments of labeled consumer products or industrial products which allow for authentication and/or determination of source. It should be appreciated that there are numerous variations and modifications of the compositions and uses described herein that are encompassed by the present invention. Accordingly, the description of a certain exemplary embodiment should not be deemed to limit the scope of the present invention.

In one aspect, the invention provides a consumer product or industrial product comprising a unique marker profile comprising one, two or more marker substances. The one, two or more marker substances comprise one, two or more polyethylene glycols which are added during the manufacturing process, i.e., at the place of origin. These marker substances can be employed in a variety of embodiments, several of which are described below, e.g., to authenticate a particular factory of manufacture, or determine the intended country or regional market for which a particular consumer product was intended.

### Use

The present invention is directed to the use of a unique marker profile for labeling consumer products or industrial products. By labeling consumer products or industrial products it is possible to indicate or trace any aspect of the consumer product or industrial product. For example, the site of manufacture of the product or the intended recipient of the product (e.g., geographical or other market for intended sale) may be indicated using the present invention. With the present invention it is also possible to use the marker as a marker of authenticity. This means that it is possible to determine whether the product is the original product or whether it has been tampered, for example by diluting a liquid product, by incorporating inferior parts into a mechanical component, or the like.

The inventive use is also suitable for determining whether a manufacturer has produced a prescribed number of products which has been labeled with the inventive marker profile or whether there has been an over production, for example in case a licensee is only allowed to produce a specific number of products.

### Marker Substances

As used herein, the term “marker” means any compound, molecule or aggregate of interest for labeling a consumer product or industrial product. In one embodiment, markers are those that may be added to a consumer product for human consumption, without requiring approval from the FDA or any other national consumer protection agency. The marker substances should be stable for an extended period, such as weeks or months or even years, in the respective consumer products or industrial products.

The term “unique marker profile” in the context of the present invention means that the marker profile is specific and is only used once, so that the combination (and amounts) of marker substances is unambiguously attributed to one specific product or to one specific use.

The unique marker profile according to the invention comprises at least one, two or more polyethylene glycols (PEG). When using PEG's, the molecular weight of the PEG's is not limited. In one embodiment the molecular weight of the PEG's is preferably more than about 100.000 Da, about 250.000 Da, about 400.000 Da, about 500.000 Da, about 1 Million or more. In another embodiment the molecular weight of the PEG's is preferably less than about 5000 Da, 4000 Da, 3000 Da, 2000 Da, 1,500 Da, 1000 Da, 900 Da, 800 Da, 700 Da, 600 Da, 500 Da, or 400 Da, and/or greater than about 100 Da, 150 Da, 200 Da, 250 Da, 300 Da, 400 Da, and 500 Da. Typically, the PEG has at least about 2, 3, 4, 5, 7, 10, 15, 20, 25, 30, 40, 50, 75, or 100 repeating monomeric units and/or not more than about 15, 25, 50, 75, 100, 150, 200, 250, or 500 repeating monomeric units.

The unique marker profile comprises at least one polyethylene glycol. Also encompassed by the present invention is the use of two or more polyethylene glycols, such as two, three, four, five, six, seven eight, nine or more different polyethylene glycols. In embodiments where the marker substances include two or more polyethylene glycols, each polyethylene glycol should be distinguishable by chemical and/or physical analysis. For example, each polyethylene glycol in the marker substance should have a different molecular weight. In this way, the identity and/or the quantity and/or the relative amounts of each polyethylene glycol in the combination of marker substances may be determined e.g., by various forms of mass spectrometry. It is possible to use mixtures of PEG's with a high molecular weight, such as for example of about 100.000 Da or more, mixtures of PEG's with low molecular weight, such as for example about 5000 Da or less, or mixtures of PEG's with all possible molecular weights, i.e. mixtures of PEG's having both high and low molecular weight. Further, in such mixtures, high molecular weight PEGs may be separated for example in classes of still polydisperse PEGs with a narrow molecular weight distribution. Those substances may be used instead of (monodisperse or polydisperse) low molecular weight compounds.

The one, two or more polyethylene glycol can be a polydisperse polyethylene glycol or a monodisperse polyethylene glycol. It is also possible to use mixtures of both polydisperse polyethylene glycols and monodisperse polyethylene glycols within one unique marker profile.

Polyethylene glycols are surprisingly suitable for labeling of consumer products or industrial products. They have shown not to alter the characteristics of the consumer product or industrial product, are chemically resistant and can be easily detected. Further, they can be used in a very low concentration (as low as 0.01 wt % and not higher than for example 1%) and still be detected by for example Raman Spectroscopy. The preferred concentration of each of the markers used is about 0.02-1 wt %, 0.05-0.8 wt %, 0.2-0.6 wt % and especially about 0.5 wt %.

In addition to the one, two or more polyethylene glycols any suitable marker substance or combination of marker substances may be used as described herein. A marker substance or combination of marker substances includes any substance or combination of substances that can be added to the intended consumer product or industrial product without affecting the behavior or usefulness of the consumer product or industrial product, for example without altering the taste, color or consistency of the product in case the product is a beverage or food, while providing a unique label associated with a particular source or intended recipient. In particular in case the product is a beverage or food product, it is important that the marker substances are soluble or miscible in or with the product so that the one, two or more marker substances are not visible as such. The detectable combination of different PEGs which can be used as markers for different liquids may be as low as 50 pg/ml and still be detectable. As explained above, the marker substances should not alter the taste of the product and should not have influence on the colour, consistence or even storage life of the product.

The marker substances are characterized in that they are detectable by known and routine detection methods already established in chemical investigation laboratories, such as for example common methods of clinical analytical chemistry. Such marker substances may or may not be taken up by the body upon administration, and are preferably recognized, for example by the FDA or any other national consumer protection agency, as acceptable inactive ingredients for beverages and/or foods.

Exemplary additional marker substances may be drawn from a number of different types of chemical species, such as but not limited to:

Carbohydrates, such as heptuloses, hexoses, pentoses, tetroses, trioses, in natural, oxidized, or reduced forms;

Disaccharides, such as lactose and chitobiose;

Tri-, tetra-, or oligosaccharides, such as N- or O-linked glycoprotein oligosaccharides,

Polysaccharides, such as starches, mannane, xylene, cellulose, hemicelluloses, and cleavage products or derivatives thereof;

Isoprenoids, such as dolichole or dolichole phosphate,

Lipids, such as triglycerides, stearines, and other fatty acids,

Steroids, such as cholesterol and its derivatives;

Acrylic polymers, such as carbomers and carboxypolymethylenes;

Poloxamers;

Polyoxyls;

Polysorbates;

acesulfame and salts thereof (e.g, acesulfame potassium)

acetylated monoglycerides

butylparaben (butyl parahydroxybenzoate)

povidone (polyvinylpyrrolidone)

copovidone (crospovidone)

gelucires (mixture of glycerides and esters of polyethylene glycol including, for example, mono-, di- and triglycerides and mono- and diesters of PEG; e.g., gelucire 33/01, 37/02, 39/01, 43/01, 44/14, 50/02, 50/13, 53/10, and 62/02)

hypromelloses (hydroxypropyl methyl cellulose)

polycarbophil (polyacrylic acid cross-linked with divinyl glycol)

polydextrose,

fluorescent dyes,

tartaric acid and salts thereof (2,3-dihydroxybutanedioic acid)

and derivatives or mixtures of these substances.

In some embodiments, the marker substances are not natural or synthetic polypeptides, nucleic acids, or other non-nucleic acid polymers that are identifiable by sequencing.

When using additional polymers as marker substances (e.g., celluloses, polyacrylic acids, polydextrose, etc.), the molecular weight of the polymers is not specifically limited. In one embodiment, the molecular weight is preferably less than about 5000 Da, 4000 Da, 3000 Da, 2000 Da, 1,500 Da, 1000 Da, 900 Da, 800 Da, 700 Da, 600 Da, 500 Da, or 400 Da, and/or greater than about 100 Da, 150 Da, 200 Da, 250 Da, 300 Da, 400 Da, and 500 Da. Typically, the polymer has at least about 2, 3, 4, 5, 7, 10, 15, 20, 25, 30, 40, 50, 75, or 100 repeating monomeric units and/or not more than about 15, 25, 50, 75, 100, 150, 200, 250, or 500 repeating monomeric units.

In embodiments where the marker substances additionally include one, two or more sugars, one, two or more sugars may be selected from the group consisting of arabinose, erythrulose, myo-inositol, cis-inositol, mannitol, sorbose, rhamnose, sorbitol, xylose and xylulose, or any other sugar which is soluble in water. Preferably, when sugars or their derivatives are used as additional marker substances, the marker substances can be easily detected by routine, known chemical evaluation techniques, such as enzymatic tests.

Various fluorescent dyes are known to the skilled person. In embodiments, where the marker substances include more than one fluorescent dyes, it is preferred if the different fluorescent dyes absorb and/or emit light at different wavelengths.

The marker substances may be used in an amount which is still detectable in diluted samples of the product and/or after a specific period of time. The amount should be such that any influence or interaction with the consumer product or industrial product can be avoided or prevented.

### Consumer Products and Industrial Products

Consumer products according to the present invention are any products which are produced and subsequently consumed by the consumer, to satisfy his current wants or needs. Such consumer products may be, but are not limited to, liquids, foods, luxury articles, consumer electronics, compact discs, documents, medical instruments, clothes, shoes, decorating materials, such as scented candles and the like, adhesives, cleaners, paint, varnish, caulk, flooring, mortar, insulation, expanded foam, cement, concrete, joint compound, tar, drywall, cosmetics and the like. In a preferred embodiment the consumer product is a liquid, cosmetic and/or food.

Industrial products according to the present invention are any products which are used for industrial purposes, i.e. products which are further processed or used to built, prepare, generate, manufacture or grow other products. Generally, such industrial products encompass all sorts of industrial markets, such as adhesives, agrochemicals, automotive and transport, building and construction, beauty and health care, dietary supplements, electronics, energy and resources, food and feed, furniture and wood, household, industrial and institutional cleaning, lubricants, oleochemicals, packaging, print and paper, paints and coating, phase change materials and thermal management, polymers, additives, skin health, textiles, nonwoven and tissues, water treatment and the like. Non-limiting examples for industrial products may be, but are not limited to, piping, plumbing fixtures, aircraft parts, motor vehicle parts, train parts, machine parts, watercraft parts, building construction components, engine components, manufactured boards, commodity chemicals, prepared chemicals such as agrochemicals, for example fertilizers, fungicides, herbicides, bactericides, crop enhancers, plant growth regulators, insecticides, insect repellants or attractants, adhesives, including liquid adhesives, foams or adhesive films or coatings, sealants, insulation foams, oleochemicals, lubricants, oils, grease, fats, and the like, fules including petrochemicals, synthetic or biosynthetic fuels, additives for lubricants or fules, surfactants, surfactants, coatings for industrial or domestic applications, such as anti-micorbial, water-repellent or anti-adhesive coatings, paper coatings, textile, coatings, such as coatings for plastics, for medicial products, (mechanical) spare parts, electronic parts, and the like. Also, agriculture products such as seeds, plants and the like are encompassed by industrial products.

The term “liquid” describes the aggregate state of the product and is not limited to a specific product sector. Thus, the term “liquid” may described product in the field of cosmetics, beverages and the like. In one embodiment the liquid is a beverage. Any beverage may be labeled with the inventive unique marker profile provided that the characteristics of the product are not negatively altered. Examples of beverages are both alcoholic and non-alcoholic beverages and may be selected from, but not limited to, water, lemonade, cola, energy drinks, juice, beer, wine, champagne, vermouth, vodka, scotch, whiskey, bourbon, brandy, cognac, gin, rum, tequila, schnapps, absinthe, akvavit, amaretto, amarula, arak, cassis, campari, contreau, curacao, drambuie, Galliano, Grand Marnier, Jagermeister, kahlua, kirsch, Midori, ouzo, pernod, sambuca, triple sec, sake and the like. In one embodiment the beverages are upscale products, such as specific brand products. Also, fuels, inks, paints and the like are encompassed by the term “liquid”.

Food according to the present invention is any substance consumed to provide nutritional support for an organism. The food may be of plant or animal origin or is processed, partially processed, or unprocessed for consumption. Non-limiting examples of food which may be labeled according to the present invention are legumes, fungi, nuts, seeds, baked goods, breads, meat, cereals, seafood, stable food, noodles, fruits, milk products, sweets, chocolate, coffee, cocoa, vegetable oils, such as palm oils, pralines and caviar or the like. There is not limitation with respect to the kind of foods.

Consumer electronics or home electronics are electronic or digital equipment intended for everyday use, typically in private homes. Consumer electronics include devices used for entertainment (flatscreen TVs, DVD players, DVD movies, iPods, video games, remote control cars, etc.), communications (telephones, cell phones, e-mail-capable laptops, etc.), and home-office activities (e.g., desktop computers, printers, paper shredders, etc.).

In one embodiment clothes and/or shoes may also be labeled with the unique marker profile of the present invention. For example, the raw material for these products may be contacted with the marker substances before the preparation of the final clothes or shoes. Examples of such raw materials are, but are not limited to, leather, wool, polyester, nylon, rubber and the like. As described below, the marker substances may also be added to these products in the form of a tag or the like.

In one embodiment cosmetics substances or products, such as beauty and personal care products, are used to enhance or alter the appearance or fragrance of the body. Examples for cosmetics include, but are not limited to, lipstick, mascara, eye shadow, foundation, rouge, skin care products, such as skin cleansers and skin lotions, hair care products, such as shampoo, hairstyling products, perfume, toothpaste and cologne. Also, basic ingredients for cosmetic applications are encompassed by this definition.

In one embodiment documents are official documents, such as from an authority, school, university and the like.

Luxury articles according to the present invention are generally articles or goods considered to be at the highest end of the market in terms of quality and price. Examples for luxury articles include, but are to limited to, haute couture clothing, accessories, and luggage, automobile, yacht, wine, bottled water, coffee, tea, foods, tobacco and tobacco products such as cigarettes and cigars, e-cigarettes or vaporizers and e-liquids, watches, clothes, jewelry, feminine hygiene products, and high fidelity.

In one embodiment the consumer product is a beverage and food.

In any of the above embodiments the consumer product is not a pharmaceutical product.

Incorporation of the Marker Substance(s) into or onto a Consumer Product

It is preferable to use the one, two or more marker substances or combination of multiple marker substances to label a particular consumer product or industrial product in a way that allows for determination of origin and/or source with a relatively high degree of certainty. In some instances, this may involve adding a single marker substance to the product during manufacture at a non-routine concentration, or adding combinations of 2, 3, 4, 5, or more marker substances, each at its own independent concentration. It is readily apparent that the more marker substances are present, each at their own independent levels of concentration, the more unlikely a particular combination is to be used as excipient in the routine manufacture of a consumer product or industrial product.

As described above, a number of different marker substances may be used in various embodiments of the invention. However, not all marker substances may be suitable for all possible consumer products or industrial products. For example, marker substances suitable for inclusion in specific beverage may not be suitable for labeling food or perfume. It is apparent to one of skill in the art which marker substances are suitable for a particular consumer product.

Once suitable marker substances have been selected, the marker substances may be incorporated to the consumer product or industrial product at the manufacturing site. These additions are typically included in a specific percentage (by weight) of the consumer product. The mechanics of these additions will vary according to the particular consumer product or industrial product being labeled and the nature and amount of the marker substances being included, but will be readily determined by those of skill in the art.

Generally, the manner of incorporation of the unique marker profile into the desired product is not critical, and thus, may be accomplished in a wide variety of ways, such as overcoating, blending, printing, and the like.

In one embodiment the unique marker profile may be incorporated to the consumer product or industrial product after the preparation or manufacturing process. This is particularly the case when the unique marker profile is attached to the outer surface of the product or to a container, vessel, tan, bowl, bottle etc. which contains the consumer product or industrial product, such as a beverage or a perfume. The unique marker profile may be attached to the outer surface of the product or the respective vessel and may be coated with a suitable coating material, for example in the form of a film, a tag, a paper wrapping or the like. The unique marker profile may also be directly incorporated into, for example, the film or tag which is attached to the respective vessel. In any of the above embodiments the unique marker profile acts as a kind of water mark.

Such attachment is also comprised for many products for which a mixing of the marker substances with any raw material during the preparation or manufacturing process is not possible. For example, CD's may be labeled by attaching the marking substances as described above. It is also possible to attach the marker substances at a “hidden” position, such as for example inside a consumer product, e.g. a consumer electronic product.

In another embodiment of the present invention the unique marker profile may be incorporated into an adhesive label, such as an adhesive tape, stickers, seals and the like. The adhesive label may then be attached to the respective consumer good or industrial product on the outside of the consumer product, industrial product or a packaging thereof or even at a hidden position within the consumer good, industrial product or packaging thereof.

In still another embodiment of the present invention documents, such as official documents, may be labeled with the unique marker profile by dipping the document or a sheet of paper which should be used for the document into a liquid preparation of the unique marker profile. After drying of the document the unique marker profile is incorporated into the document as a kind of watermark.

In this regard it is noted that the term “in the product” as used herein may also mean “on the product” in case of attaching the unique marker profile on the consumer product, such as a vessel, packaging or the like.

Methods of Identifying the Source and/or Intended Recipients of a Consumer Product

The number of marker substances and amount of each included in a consumer product or industrial product may be varied to achieve any of the uses described herein. For example, each of plurality of manufacturing facilities may be assigned a unique marker profile (i.e., a unique set of marker substances, optionally including the absolute or relative concentrations of each marker substance) that is incorporated into consumer products or industrial products manufactured or produced at that facility. In this way, the particular manufacturing plant or place of production of any individual consumer product (i.e., a consumer product's place of origin) may later be identified by determining the identities (and possibly amounts) of marker substances contained in the consumer product or industrial product and comparing this information to known marker profiles for each possible manufacturing or production facility. Such a determination facilitates authentication of a consumer product, and thus aids in identification of counterfeit consumer products or industrial products. In some embodiments specific amounts of the marker substances or a specific amount of the marker profile is used to provide additional information about the product and to add an additional layer of security. For example, in using a specific amount of one, two or more marker substances, it is possible to determine whether the product has been tempered, for example by dilution or any other means as the amount of marker substances in a diluted product would differ to the amount in the original product.

A larger number of unique marker profiles may be generated from a smaller number of marker substances by varying the relative concentrations of the marker substances in known/pre-determined ratios. For example, in its simplest form when only two marker substances are used, represented generically as X and X′, multiple marker profiles may be constructed by varying the ratios of X to X′ as follows:

| | Relative Amount of | |
|---|---|---|
| | Marker Substance | |
| Marker # | X | X′ |
| 1 | 1 | 0 |
| 2 | 0 | 1 |
| 3 | 1 | 1 |
| 4 | 1 | 2 |
| 5 | 1 | 3 |
| 6 | 1 | 4 |
| 7 | 2 | 1 |
| 8 | 3 | 1 |
| 9 | 4 | 1 |

The ratios of the paired marker substances are not limited to those ratios shown in the table above but may include any convenient ratio or combination of ratios such as 5:1, 10:1, 15:1, 20:1, or more. The only practical limits are those of convenience and detectability. Furthermore, the strategy of constructing unique marker profiles based on the relative ratios of marker substances is not limited to pairs of marker substances but instead can be extended to varying the relative ratios of 3, 4, 5, 6, 7, 8, or more marker substances, thereby significantly increasing the total number of unique marker profiles available for any given number of marker substances, without increasing the number of chemically distinct marker substances.

In some embodiments, every intended recipient of a consumer product or industrial product may be assigned a unique marker profile (i.e., a unique set of marker substances, optionally including the absolute or relative concentrations of each marker substance) that is incorporated into the consumer product or industrial product during manufacture. Later identification of this unique marker profile in a particular consumer product or industrial product allows for determination of the intended recipient of the consumer product or industrial product.

In some embodiments, all intended recipients meeting a particular criterion (e.g., those within a certain geographic region, or those subject to a common set of laws and/or regulations) are assigned a unique marker profile (i.e., a unique set of marker substances, optionally including the absolute or relative concentrations of each marker substance) that is incorporated into the consumer product or industrial product during manufacture. Thus, consumer products or industrial products made for all intended recipients meeting a particular criterion include a unique marker. Later identification of this unique marker profile in a particular product allows for determination of the group of intended recipients that share a preselected criterion.

It should be understood that in some embodiments, the identity of a marker substance and the quantity of that marker substance may each be used to convey different information. The following example is not intended to be limiting and is intended to illustrate one possible embodiment. Take for example a scenario where some particular consumer product is prepared by a Manufacturing Facility 1 (for example a manufacturing facility in the USA) with two different intended recipients. Manufacturing Facility 1 may be assigned a unique marker profile comprising two marker substances, A and B. Thus, all products manufactured at Manufacturing Facility 1 include both marker substances A and B. However, the amounts (absolute or relative) of the two marker substances may be varied to indicate the intended recipient. For example, products manufactured for intended recipient 1 (for example the German market) may comprise some amount of marker substance A, and twice that amount of marker substance B. Products manufactured for intended recipient 2 (for example the UK market) may comprise some amount of marker substance A, and one half that amount of marker substance B. In this way, later analysis of the identity of the marker substances allows for determination of the source of the product (i.e., Manufacturing Facility 1 because of the presence of both marker substances A and B), and analysis of the relative amounts of the marker substances allows for the determination of the intended recipient of the product.

In other embodiments, each piece of information that is to be conveyed by the marker substances is encoded with at least one different marker substance. The following illustrative example is provided to contrast the example described above. Consider the same scenario as above where some particular consumer product is prepared by Manufacturing Facility 1 with two different intended recipients. Manufacturing Facility 1 may be assigned a unique marker profile comprising two marker substances, A and B. Thus, all products manufactured at Manufacturing Facility 1 include both marker substances A and B. However, in these embodiments, one, two or more additional marker substances are used to encode the identity of the intended recipients. For example, products manufactured for intended recipient 1 may comprise some amount of an additional marker substance C, while products manufactured for intended recipient 2 may comprise some amount of additional marker substances D and E. In this way, later analysis of the identity of the marker substances in a product from this facility would show the presence of marker substances A and B, and either C or D and E. The identification of marker substances A and B allows for determination of the source of the product (i.e., Manufacturing Facility 1), and identification of marker substances C or D and E allows for the determination of the intended recipient of the product.

In other embodiments, the marker profile can be used to determine whether the product is the correct product from a variety of products. This may be crucial as the use of the wrong product may have detrimental consequences, such as in case of fertilizers, fungicides or the like. For example, a Manufacturing Facility may be assigned a unique marker profile comprising two marker substances, A and B. Each product from this facility has additional markers to uniquely encode each product. In this way, later analysis of the identity of the marker substances in a product from this facility would show the presence of marker substances A and B, and each specific product would show the presence of the specific unique marker profile. This allows the determination of each product and avoids the use of the wrong product for a specific use.

The above illustrative examples demonstrate that in some embodiments a plurality of marker substances may be used. In fact, the above examples may be considered to be relatively simple, in as far as the number of manufacturing facilities and intended recipients in the examples are very limited. However, it should be appreciated that the number of marker substances that can be used in any given unique marker profile is only limited by the number of suitable marker substances available. It should also be appreciated that a unique marker profile may contain as few as a single marker substance, with or without consideration of its relative or absolute concentration in the consumer product or industrial product.

It is preferable to include multiple marker substances in a consumer product or industrial product, wherein it is possible by the combination of marker substances to develop a certain numerical code belonging to a respective sample. For example, it is preferred to include a combination of at least 2, such as at least 3, such as at least 4, such as at least 5 marker substances simultaneously. Using a total of n marker substances, there exist 2ⁿ⁻¹ different combinations in a dual numeric system; that is without use of absolute or relative concentrations of each marker as additional indicia.

In some embodiments, a unique marker profile comprises a plurality of marker substances, and two or more of the plurality of marker substances are of the same type of marker substances (e.g. two or more polyethylene glycols, or in addition to the polyethylene glycol(s) two or more sugars, two or more isoprenoids, two or more lipids, two or more saccharides, two or more starches, two or more polyols, etc. as described elsewhere). In some related embodiments, every marker substance in the plurality of marker substances is of the same type of marker substance.

In some embodiments a unique marker profile comprises a plurality of marker substances, and two or more of the plurality of marker substances are different types of marker substances (e.g. besides the one, two or more polyethylene glycols, one sugar, one lipid, one isoprenoid, one starch, etc. as described elsewhere). In some related embodiments, no two marker substances in the plurality of marker substances are of the same type of marker substance.

In some embodiments a unique marker profile comprises three or more marker substances, and two or more marker substances are of the same type of marker substance, while at least one of the three or more is of a different type of marker substance.

Thus, the unique marker profile of the invention allows to identify the manufacturing facility and/or the country of origin, allows to identify the specific market or region for which the product is intended and allows to identify whether the product is a counterfeit product or an original product.

A number of enzymatic, immunological, mass spectrometric and electrophoretic detection methods as well as combinations of these methods are available for determining the identity and/or quantity and/or relative amounts of at least one marker substance in a consumer product, industrial product or sample thereof. Preferably, analysis is conducted by an instrumental analytical chemistry technique, such as coupled Gas Chromatography/Mass Spectrometry (GC/MS) (with single or tandem MS), High Performance Liquid Chromatography/Mass Spectrometry (HPLC/MS) (with single or tandem MS), High Performance Liquid Chromatography (HPLC), Gas Chromatography (GC), capillary electrophoresis, thin-layer chromatography, gel electrophoresis, column chromatography, and the like. These methods allow the very time-efficient investigation of, in particular, liquid samples or of samples which, due to their preparation were transferred into a liquid. At the same time, these detection methods allow a high degree of automation so that a multitude of consumer products or industrial products or samples can be analyzed in a short time and, in as far the chromatograms and, as the case may be, mass spectroscopic fractionation patterns of reference substances already exist in the computer evaluation unit, the analysis of the at least one marker substance is also greatly simplified.

In another embodiment, an analytical method may be performed in the field with an immediate yes/no readout obtained. Such methods may, for example, employ a “dip-stick” type of analysis. For example, a portable analytical apparatus (e.g. Raman spectrometer) may be used to get an immediate result and to take appropriate measures, such as confiscating the product. The consumer may be provided with a suitable analytical apparatus to verify the origin of the product. For example, it might be necessary to verify that a product which has been returned by a costumer is genuine and has not been altered.

### EXAMPLES

### Example 1

As an example, an embodiment for uniquely marking of a liquid consumer product to enable later identification of the manufacturing facility of origin is provided below.

A particular liquid consumer product is manufactured at three manufacturing facilities: Facility 1, Facility 2, and Facility 3. Each manufacturing facility is assigned a unique marker profile comprising two marker substances. These two marker substances are added in equal amounts during the manufacture of the liquid consumer product, such that the total concentration of the marker substances is about 1% of the liquid product.

At Facility 1, the liquid product is produced such that the product comprises about 0.5% wt of Marker A, a monodisperse polyethylene glycol with an approximate molecular weight of about 530 amu, and about 0.5% wt of Marker B, a monodisperse polyethylene glycol with an approximate molecular weight of about 574 amu.

At Facility 2, the liquid product is produced such that the product comprises about 0.5% wt of Marker A, and about 0.5% wt of Marker C, a monodisperse polyethylene glycol with an approximate molecular weight of about 618 amu.

At Facility 3, the liquid product is produced such that the product comprises about 0.5% wt of Marker B, and about 0.5% wt of Marker C.

After production, the products are distributed to wholesale suppliers, and ultimately to approved retail locations, where the products are available for purchase. If a liquid product purported to be genuine is later found at an unapproved retail location, the responsible law enforcement or regulatory body may send a sample of the purportedly genuine liquid product for mass spectrometric analysis for identification and quantitation of marker substances present in the product.

Several possible results of such analysis, and their meanings with respect to origin are presented below in Table 1.

TABLE 1

| Possible Outcome of | |
|---|---|
| Mass Spectrometric Analysis | Origin |
| Markers A and B, at about 1:1 | Facility 1 |
| Markers A and C, at about 1:1 | Facility 2 |
| Markers B and C, at about 1:1 | Facility 3 |
| No Markers present | Unknown (counterfeit) |
| Single Marker present | Unknown (counterfeit) |
| Two Markers present, but not at about 1:1 | Unknown (counterfeit) |
| Three or more Markers present | Unknown (counterfeit) |

### Example 2

As an example, an embodiment for uniquely marking of a liquid product to enable later identification of the manufacturing facility of origin, and intended recipient is provided below.

A particular liquid product is manufactured at two manufacturing facilities: Facility 1 and Facility 2. There are two intended recipients for the products, and each intended recipient may receive products from either Facility. The intended recipients for the product are Intended Recipient 1 and Intended Recipient 2.

Each combination of manufacturing facility and intended recipient is assigned a unique marker profile comprising four marker substances. Two marker substances are added in equal amounts during the manufacture of the liquid product, such that the total concentration of these two marker substances is, for example, about 1% of the liquid product. Two other marker substances are added at a ratio of 2:1 during the manufacture of the liquid product, such that the total concentration of these marker substances is, for example, about 1.5% of the liquid product. Thus, the total concentration of the four marker substances is about 2.5% of the total liquid product.

At Facility 1, all liquid products, regardless of intended recipient, are produced such that the product comprises about 0.5% wt of Marker A, a monodisperse polyethylene glycol with an approximate molecular weight of about 530 amu, and about 0.5% wt of Marker B, a monodisperse polyethylene glycol with an approximate molecular weight of about 574 amu.

At Facility 2, all liquid products, regardless of intended recipient, are produced such that the product comprises about 0.5% wt of Marker A, and about 0.5% wt of Marker C, a monodisperse polyethylene glycol with an approximate molecular weight of about 618 amu.

All liquid products intended for Intended Recipient 1, regardless of production facility, are produced such that the product comprises about 0.5% wt of Marker D, a monodisperse polyethylene glycol with an approximate molecular weight of about 662 amu, and about 1% wt of Marker E, a monodisperse polyethylene glycol with an approximate molecular weight of about 706 amu.

All liquid products intended for Intended Recipient 1, regardless of production facility, are produced such that the product comprises about 1% wt of Marker D, and about 0.5% wt of Marker E.

After production, the products are distributed to wholesale suppliers, and ultimately to approved retail locations, where the products are available for purchase. If a liquid product purported to be genuine is later found at an unapproved retail location, the responsible law enforcement or regulatory body may send a sample of the purportedly genuine liquid product for mass spectrometric analysis.

Several possible results of such analysis, and their meanings with respect to origin and intended recipient are presented below in Table 2.

TABLE 2

| Possible Outcome of | | Intended |
|---|---|---|
| Mass Spectrometric Analysis | Origin | Recipient |
| Markers A, B, D, E; present at about 1:1:1:2 | Facility 1 | Intended Recipient |
| | | 1 |
| Markers A, B, D, E; present at about 1:1:2:1 | Facility 1 | Intended Recipient |
| | | 2 |
| Markers A, C, D, E; present at about 1:1:1:2 | Facility 2 | Intended Recipient |
| | | 1 |
| Markers A, C, D, E; present at about 1:1:2:1 | Facility 2 | Intended Recipient |
| | | 2 |
| No Markers present | Unknown (counterfeit) | N/A |
| Single Marker present | Unknown (counterfeit) | N/A |
| Two Markers present | Unknown (counterfeit) | N/A |
| Three Markers present | Unknown (counterfeit) | N/A |
| Four Markers present, but wrong combination | Unknown (counterfeit) | N/A |
| Four Markers present, but at incorrect ratio | Unknown (counterfeit) | N/A |
| Five or more Markers present | Unknown (counterfeit) | N/A |

If the product under investigation is found to lack Markers A and B, or A and C, then the product is known to be counterfeit.

If the product is validated as genuine as to source but is found on the black or grey market, knowledge of the intended recipient, and thus the intended distribution network through which that product was intended to proceed, may prove useful in investigating the break in the supply chain which lead to availability of the product on the black or grey market.

### Example 3

As an example, an embodiment for uniquely marking of an agri-product (or agro-product) to enable later identification of the manufacturing facility of origin, and intended recipient is provided below.

A fungicide for use for specific crops is manufactured at a specific manufacturing facility. The manufacturing facility is assigned a unique marker profile comprising two marker substances selected from different polyethylene glycols. The specific fungicide is produced such that it comprises about 0.5% wt of another Marker A and about 0.5% wt of another Marker B.

The consumer is now in the position to identify the marker profile with an appropriate analyzing system (which may also be provided by the manufacturer) in order to ensure that a) the product is indeed from the manufacturer and b) that the fungicide is the correct fungicide for the crop to ensure the right product is used on the right crop to avoid damage and insurance claims.

### Example 4

As an example, an embodiment for uniquely marking of a liquid product to enable later identification of the manufacturing facility of origin, and to identify whether the product has been tampered.

A particular liquid product is manufactured at a manufacturing facility. The manufacturing facility is assigned a unique marker profile comprising three marker substances. The marker substances are added in a specific amount during the manufacture of the liquid product, such that it is known how much marker is present in the total amount of the liquid and how much should be present in a sample thereof. The marker substances can be three different PEGs each with different molecular weight. The used marker substances are preferably a mixture of low molecular PEGs. The amount of each PEGs used should be less than 1 mg/ml, more preferably 1 ng/ml and even more preferably less than 100 pg/ml.

In case the liquid product is diluted later and sold as an alleged original product, it is possible to determine the amount of marker substance present in the liquid. In case the determined amount is corresponding to the amount which has been added at the manufacturing facility, the liquid is the original product, whereas in case the amount determined is less than the originally added amount or not detectable because of the detection limit of the marker substances, it can be concluded that the liquid has been diluted or otherwise altered and does no longer correspond to the original product.

### Example 5

As an example, an embodiment for uniquely marking a consumer product to enable later identification of the manufacturing facility of origin and to identify a respective batch.

A licensee is limited to the production of a batch size of 100.000 units of a specific consumer product, wherein the overall amount of marker substances is adapted to this particular batch-size. In case of an overproduction the product will either not be marked at all as no marker substances would be left for marking the product or the marker has been diluted to also mark the over produced products. Both can be easily determined.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

Terms and phrases used in this document, and variations thereof, unless otherwise expressly stated, should be construed as open ended as opposed to limiting. As examples of the foregoing, the term ‘including’ should be read to mean ‘including, without limitation’ or the like; the term ‘comprising’ as used herein is synonymous with ‘including,’ ‘containing,’ or ‘characterized by,’ and is inclusive or open-ended and does not exclude additional, unrecited elements or method steps; the term ‘example’ is used to provide exemplary instances of the item in discussion, not an exhaustive or limiting list thereof; and adjectives such as ‘known,’ ‘normal,’ ‘standard,’ and terms of similar meaning should not be construed as limiting the item described to a given time period or to an item available as of a given time, but instead should be read to encompass known, normal, or standard technologies that may be available or known now or at any time in the future. Likewise, a group of items linked with the conjunction ‘and’ should not be read as requiring that each and every one of those items be present in the grouping, but rather should be read as ‘and/of’ unless expressly stated otherwise. Similarly, a group of items linked with the conjunction ‘or’ should not be read as requiring mutual exclusivity among that group, but rather should be read as ‘and/or’ unless expressly stated otherwise. In addition, as used in this application, the articles ‘a’ and ‘an’ should be construed as referring to one, two or more than one (i.e., to at least one) of the grammatical objects of the article. By way of example, ‘an element’ means one element or more than one element.

The presence in some instances of broadening words and phrases such as ‘one, two or more,’ ‘at least,’ ‘but not limited to,’ or other like phrases shall not be read to mean that the narrower case is intended or required in instances where such broadening phrases may be absent.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1. Use of a unique marker profile for labeling consumer products or industrial products, wherein the unique marker profile comprises one, two or more marker substances comprising one, two or more polyethylene glycols and wherein the products comprise at least one product selected from the group consisting of liquids, food, luxury articles, consumer electronics, compact discs, documents, medical instruments, clothes, shoes, decorating materials, adhesives, cleaners, engine components, paint, coating, varnish, caulk, flooring, mortar, insulation, expanded foam, cement, concrete, joint compound, tar, drywall, piping, plumbing fixtures, aircraft parts, motor vehicle parts, train parts, machine parts, watercraft parts, building construction components, engine components, manufactured boards, commodity chemicals, prepared chemicals, agrochemicals, mechanical, spare parts, electronic parts, and cosmetics.

2. The use of claim 1, wherein the one, two or more marker substances further comprise one, two or more marker substances selected from the group consisting of carbohydrates, disaccharides, trisaccharides, tetrasaccharides, oligosaccharides, polysaccharides, isoprenoids, lipids, steroids, acrylic polymers, poloxamers, polyoxyls, polysorbates, acesulfame, an acetylated monoglyceride, butylparaben, povidone, copovidone, crospovidone, gelucire, hypromelloses, polycarbophil, polydextrose, tartaric acid or a salt thereof, and derivatives thereof.

3. The use of claim 1, wherein said one, two or more marker substances are substances identifiable or quantifiable by an enzymatic, immunological, spectrometric, or electrophoretic method.

4. The use of claim 1, wherein said one, two or more marker substances are identifiable or quantifiable by Gas Chromatography/Mass Spectrometry (GC/MS), Gas Chromatography/Tandem Mass Spectrometry (GC/MS/MS), High Performance Liquid Chromatography/Mass Spectrometry (HPLC/MS), High Performance Liquid Chromatography/Tandem Mass Spectrometry (HPLC/MS/MS), High Performance Liquid Chromatography (HPLC), Gas Chromatography (GC), or Raman Spectroscopy.

5. The use of claim 1, wherein said unique marker profile comprises two or more marker substances, and at least two of said marker substances are the same type of marker substances or
wherein at least two of said marker substances are different types of marker substances.

6. The use of claim 1, wherein the one, two or more polyethylene glycols is a polydisperse polyethylene glycol or a monodisperse polyethylene glycol.

7. The use of claim 1, wherein said unique marker profile comprises one, two or more marker substances and identities of the one, two or more marker substances indicate the origin of said consumer product or
wherein said unique marker profile comprises two or more marker substances and identities and absolute or relative amounts of the two or more marker substances indicate the origin of said consumer product, and/or
wherein said unique marker profile comprises one, two or more marker substances and identities of the one, two or more marker substances indicate the intended recipient of said consumer product, or
wherein said unique marker profile comprises two or more marker substances and identities and absolute or relative amounts of the two or more marker substances indicate the intended recipient of said consumer product, and/or
wherein said unique marker profile comprises one, two or more marker substances and identities of the one, two or more marker substances indicate a preselected characteristic of an intended recipient of said consumer product, or
wherein said unique marker profile comprises two or more marker substances and identities and absolute or relative amounts of the two or more marker substances indicate a preselected characteristic of an intended recipient of said consumer product.

8. The use of claim 1, wherein the marker substances are used in a specific amount.

9. The use of claim 1, wherein the liquid is selected from the group consisting of water, lemonade, cola, energy drinks, juice, beer, wine, champagne, vermouth, vodka, scotch, whiskey, bourbon, brandy, cognac, gin, rum, tequila, schnapps, absinthe, akvavit, amaretto, amarula, arak, cassis, campari, contreau, curacao, drambuie, Galliano, Grand Marnier, Jagermeister, kahlua, kirsch, Midori, ouzo, pernod, sambuca, triple sec and sake.

10. The use of claim 1, wherein the food is selected from the group consisting of legumes, fungi, nuts, seeds, baked goods, breads, meat, cereals, seafood, stable food, noodles, fruits, milk products, sweets, chocolate, coffee, cocoa, vegetable oils, such as palm oils, pralines, and caviar.

11. The use of claim 1, wherein the cosmetics are selected from the group consisting of lipstick, mascara, eye shadow, foundation, rouge, skin cleansers and skin lotions, shampoo, hairstyling products, perfume, and cologne.

12. A Method for labeling a consumer or industrial product including the steps of:
bringing a combination of two, three, four, five six, seven, eight, nine or more polyethylene glycols (PEGs), each of a different molecular weight, into contact with the consumer or industrial product before packaging the consumer or industrial product, to label the consumer or industrial product;
packaging the consumer or industrial product labeled with the combination of two, three, four, five six, seven, eight, nine or more PEGs;
shipping the consumer or industrial product; and
detecting the presence and/or the amount of the combination of the PEGs which were brought into the contact with the consumer or industrial product in or on the consumer or industrial product.

13. The method of claim 12, wherein the combination of two, three, four, five six, seven, eight, nine or more PEGs, each of a different molecular weight, is saved in a databank.

14. The method of claim 13, wherein the databank is part of a blockchain system.

15. The method of claim 12, wherein the detection is done by a Raman spectrometer.

16. The method of claim 15, wherein the Raman spectrometer is a hand held device.

17. A labeled consumer product or industrial product comprising:
a unique marker profile encoding one, two or more of the origin of the product, the intended recipient of the product, and a preselected characteristic of an intended recipient of the product;
wherein said unique marker profile comprises one, two or more polyethylene glycols.

18. The labeled consumer or industrial product of claim 17, wherein said one, two or more marker substances further comprise one, two or more of carbohydrates, disaccharides, trisaccharides, terrasaccharides, oligosaccharides, polysaccharides, isoprenoids, lipids, steroids, acrylic polymers, poloxamers, polyoxyls, polysorbates, acesulfame, an acetylated monoglyceride, butylparaben, povidone, copovidone, crospovidone, gelucire, hypromelloses, polycarbophil, polydextrose, tartaric acid or a salt thereof, and derivatives thereof.

19. The labeled consumer or industrial product of claim 17, wherein the one, two or more polyethylene glycols comprise one, two or more monodisperse or polydisperse polyethylene glycols.

20. The labeled consumer or industrial product of claim 17, wherein at least one of said marker substances encodes the origin of said labeled consumer or industrial product.

21. The labeled consumer or industrial product of claim 17, wherein at least one of said marker substances encodes the intended recipient of said labeled consumer or industrial product, and/or wherein at least one of said marker substances encodes a preselected characteristic of an intended recipient of said labeled consumer or industrial product.
